(19) United States
(12) Patent Application Publication
(10) Pub. No.: US 2009/0216002 A1
(43) Pub. Date: Aug. 27, 2009

(54) SYNTHESIS OF HYDROQUINONE DERIVATIVES

(75) Inventors: Stefan Kleinebekel, Bielefeld (DE); Nasir Hayat, Bielefeld (DE); Stephan Seck, Halle/Westfalen (DE); Alfred Olbrich, Halle/Westfalen (DE)

Correspondence Address: CONNOLLY BOVE LODGE & HUTZ, LLP, P O BOX 2207, WILMINGTON, DE, 19899, US

(73) Assignee: Girindus AG, Bergisch-Gladbach (DE)

(21) Appl. No.: 11/917,615

(22) PCT Filed: Jun. 14, 2006
(86) PCT No.: PCT/EP2006/063194

§ 371 Date: Jan. 20, 2009

Related U.S. Application Data
(60) Provisional application No. 60/692,248, filed on Jun. 21, 2005.

(30) Foreign Application Priority Data
Jun. 16, 2005 (EP) 05105307.2

(51) Int. Cl. C07H 1/00

(52) U.S. Cl. 536/18.6

(57) ABSTRACT

A method for synthesising a compound having the general formula (I) wherein R represents substituents selected independently from the group of H, F, Cl, Br, NO₂, alkyl, aryl, aralkyl, alkoxy, aryloxy, aralkoxy and disubstituted amine R′ is independently selected from the group of H, F, Cl, Br, OH, NO₂, alkyl, aryl, aralkyl, alkoxy, aryloxy, aralkoxy and disubstituted amine, n is selected from 1, 2 or 3.

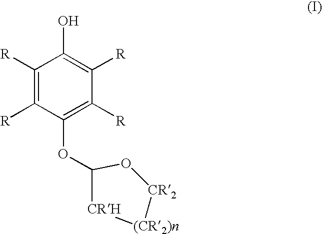

The present invention is directed to the synthesis of compounds having the general formula I

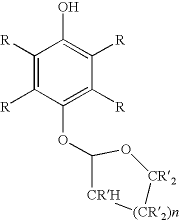

WO 95/23780 describes compounds useful for the inhibition of tyrosinase in melanosomes of epidermal melanocytes and the use as skin lightening compositions. Two of these compounds are known under the names arbutin and deoxyarbutin (4-[(tetrahydro-2H-pyran-2-yl)oxy]phenol). The application of the compounds and the use for the preparation of cosmetic or pharmaceutical compositions is described in the above-mentioned WO 95/23780, incorporated by reference.

The synthesis of these compounds, especially deoxyarbutin is described in more details in U.S. Pat. No. 5,585,525, incorporated by reference. The synthesis is based on a two step procedure. In a first step a protected intermediate is prepared through coupling of a hydroquinone derivative with an enol ether in the presence of an acid. As suitable solvents the document mentions methylene chloride, diethyl ether, tetrahydrofuran, dioxan and mixtures thereof, with methylene chloride being preferred. The product is then recristallized from, for example, hexane.

The mentioned solvents are hazardous solvents and are problematic in scale up.

The second step comprises the removal of the hydroxy protecting group, typically using hydrazine. Hydrazine hydrate is known to be carcinogenic. For use as a cosmetic or pharmaceutical product it would be absolutely necessary to avoid the presence of even traces of hydrazine hydrate in the final product.

Therefore, there is still a need to develop an improved process which avoids the difficulties and drawbacks of prior art.

According to one embodiment of the invention, there is provided a method for synthesizing a compound having the general formula

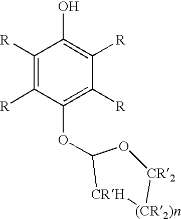

wherein

R represents substituents selected independently from the group of H, F, Cl, Br, NO₂, alkyl, aryl, aralkyl, alkoxy, aryloxy, aralkoxy and disubstituted amine

R′ is independently selected from the group of H, F, Cl, Br, HO, NO₂, alkyl, aryl, aralkyl, alkoxy, aryloxy, aralkoxy and disubstituted amine,

n is selected from 1, 2 or 3

comprising the steps of

a) reacting in the presence of an acid

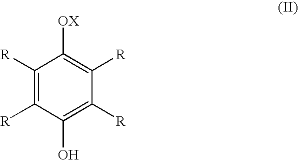

wherein X is a hydroxyl protecting group and R is as defined above with

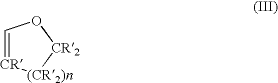

wherein R′ and n are as defined above in the presence of less than 50% by weight of a solvent

b) removing the hydroxyl protecting group.

Whereas prior art teaches the use of large amounts of solvents (the total reactant concentration is described as being between 5 to 50% by weight), it has now be found that the amount of solvent can be significantly reduced or solvent can be completely avoided.

In preferred embodiments the amount of solvent is less than 30%, preferably less than 10% and more preferably less than 5% by weight. It is especially preferred that the amount of solvent is less than 1% or that no solvent is used. The amount of solvent is calculated on a weight basis relative to the weight of compounds II and III.

Preferred acids used in the synthesis are selected from the group consisting of hydrochloric acid, sulphuric acid, phosphoric acid, toluene sulphonic acid, methane sulphonic acid and citric acid.

As a hydroxy protecting group benzyl, substituted benzyl or allyl are especially suitable.

In a preferred embodiment compound III has a melting point below 50° C.

In a further preferred embodiment, the hydroxyl protecting group is benzyl or substituted benzyl and is subsequently removed with hydrogen.

By using less and especially very small amounts or no solvent, problems with scale up can be significantly reduced. Especially the amount of hazardous solvents can be avoided when compound III functions as a solvent. If at all relatively unproblematic solvents can be added such as isopropanol, methyl tetrabutyl ether or the like.

In a second embodiment, the present invention is directed to a method for synthesizing a compound having the general formula

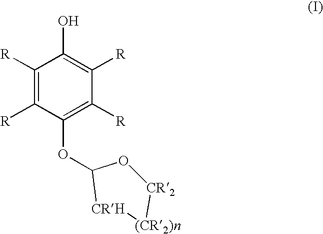

wherein

R represents substituents selected independently from the group of H, F, Cl, Br, NO₂, alkyl, aryl, aralkyl, alkoxy, aryloxy, aralkoxy and disubstituted amine

R′ is independently selected from the group of H, F, Cl, Br, OH, NO₂, alkyl, aryl, aralkyl, alkoxy, aryloxy, aralkoxy and disubstituted amine,

n is selected from 1, 2 or 3

comprising the steps of

a) reacting in the presence of an acid

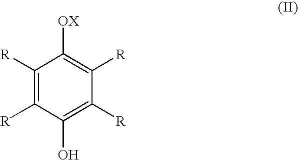

wherein X is a hydroxyl protecting group and R is as defined above with

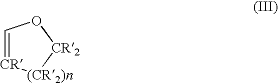

wherein R′ and n are as defined above

b) removing the hydroxyl protecting group with hydrogen and a catalyst in the presence of a solvent and a base, said base having a solubility at 20° C. of less than 5% by weight in the solvent.

It was observed that during hydrogenation the product undergoes partial cleavage of the pyran or furan group. Surprisingly, this could be prevented by adding a base which is only slightly soluble in the solvent. Typically, the base should have a solubility of less than 5% by weight of the solvent, preferably less than 3% by weight of the solvent and more preferably less than 1% by weight of the solvent. Suitable bases are, for example, alkali and earth alkali carbonates such as CaCO₃, NaHCO₃, KHCO₃, Na₂CO₃, K₂CO₃.

Suitable solvents are solvents, which are inert to hydrogenation, for example alcohols, ethers and esters which can be branched, unbranched or cyclic. Especially suitable solvents are methanol, ethanol, isopropanol, tetrahydrofuran, diethyl ether, methyl tetrabutyl ether, ethyl acetate, butyl acetate and mixtures thereof.

Hydrogenation is accomplished in the presence of a catalyst. Preferred catalysts are based on precious metals, the most preferred catalyst being palladium on carbon or other supports such as silica or aluminum oxide.

In a preferred embodiment, the synthesis makes use of both embodiments of the invention, i.e. avoids or reduces the amount of solvent in the first step and uses hydrogen and a catalyst in the presence of a base in the second step.

In very preferred embodiments of the present invention, it is applicable to compounds wherein R is H, all R′ are H and n=2, thus the resulting product being deoxyarbutin.

The invention is explained in more details by the following examples.

### Example 1

Step 1

To a stirred solution (without solvent) of 4-(benzyloxy)phenol (20 g, 0.1 mol) and 3,4-dihydropyran (23.5 g, 0.28 mol) was added concentrated hydrochloric acid (0.05 ml). The reaction mixture was stirred at 20° C. for 4 h. TLC Analysis showed only traces of starting material. A solution of 1 M NaOH (20 ml) was added to neutralize the mixture, keeping the temperature between 20 and 30° C. The mixture was stirred for 10 min and isopropanol was added and again it was stirred for 10 min. Finally, the product was filtered and washed with water (2×22 ml). The solid was suspended in water (60 ml), isopropanol (25.5 ml) and 1 M NaOH (2.5 ml) and stirred for 15 min. The suspension was filtered and washed with water (10 ml). The solid product was dried in vacuum at 40° C. and gave 76% yield and 99.5% of HPLC purity (area).

### Example 2

Step 2

To a stirred solution of the product (30 g, 0.105 mol), calcium carbonate (2.79 g, 0.027 mol) and ethyl acetate (120 ml) was added palladium on carbon (3.6 g). The mixture was stirred at ambient temperature under hydrogen pressure until uptake of hydrogen ceased. The mixture was filtered and washed with ethyl acetate. The solvent was distilled off in vacuum. The evaporation residue was dissolved in alcohol (36 ml) and the product precipitated by the addition of water (144 ml). The slurry was cooled to 0° C. and stirred vigorously for 2 h. Precipitated product was filtered and washed with water, dried in vacuum to give 80% of Deoxyarbutin with a purity of 99.8% (area by HPLC).

According to this procedure several other bases were tested (see table).

| | | | | Puritiy |
|---|---|---|---|---|
| Ex- | Size | | | HPLC |
| ample | (g) | Target | Yield | (% area) |
| 3 | 30 | Ethanol in ethyl acetate CaCO₃ | 15.5 g | 99.8 |
| | | as a base | 80% | |
| 4 | 10 | Ethanol as a solvent and CaCO₃ | 5.3 g | 98.5 |
| | | as base | 78% | |
| 5 | 10 | Isopropanol as a solvent and | 5.2 g | 97.7 |
| | | CaCO₃ as a base | 77% | |
| 6 | 5 | Isopropanol as a solvent (1:7) | 5.4 g | 92.9 |
| | | and Na₂CO₃ as base | 80% | |
| 7 | 28 | Reaction with aluminium oxide | 16.1 g | 95.3 |
| | | and ethyl acetate as solvent | 82% | |

### Comparative Example 1

To a stirred suspension of the product of example 1 (5 g, 0.017 mol) and ethanol (50 ml) was added 5% palladium on charcoal with 50% water content (0.6 g). The mixture was stirred at 60° C. under hydrogen gas atmosphere for 2 h. TLC showed a side product. It was cooled to 20° C. and filtered over a small bed of diatomaceous earth, washed with alcohol. The solvent was distilled in vacuum at 40° C. The oil was dissolved in alcohol (5 ml) and heated to 80° C. To this mixture water (30 ml) was added in 30 min time and the mixture was stirred for 25 min. The mixture was cooled to 20° C. and stirred vigorously for 6 h. Precipitated product was filtered and washed with water, dried in vacuum at 40° C. to gave 0.4 g of a solid. No product was detectable by HPLC.

1-13. (canceled)

14. A method for synthesising a compound having the general formula 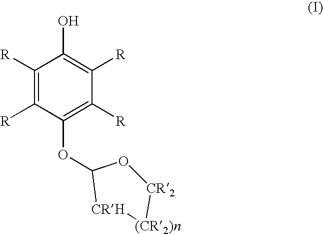
wherein
R is selected independently from the group of H, F, Cl, Br, NO₂, alkyl, aryl, aralkyl, alkoxy, aryloxy, aralkoxy, and disubstituted amine;
R′ is selected independently from the group of H, F, Cl, Br, OH, NO₂, alkyl, aryl, aralkyl, alkoxy, aryloxy, aralkoxy, and disubstituted amine; and
n is 1, 2, or 3;
comprising
a) reacting a compound of formula (II) in the presence of an acid 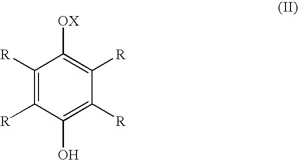
wherein X is a hydroxyl protecting group and R is as defined above;
with a compound of formula (III) 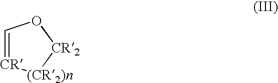
wherein R′ and n are as defined above
together with less than 50% by weight of a solvent; and
b) removing the hydroxyl protecting group.

15. The method of claim 14, wherein said acid is selected from the group consisting of hydrochloric acid, sulphuric acid, phosphoric acid, toluene sulphonic acid, methane sulphonic acid, and citric acid.

16. The method of claim 14, wherein the hydroxyl protecting group is benzyl, substituted benzyl, or allyl.

17. The method of claim 14, wherein the solvent is less than 30% by weight.

18. The method of claim 14, wherein the solvent is less than 10% by weight.

19. The method of claim 14, wherein the solvent is less than 5% by weight.

20. The method of claim 17, wherein the solvent is less than 1% by weight.

21. The method of claim 14, wherein the hydroxyl protecting group is benzyl or substituted benzyl and is removed with hydrogen.

22. The method of claim 14, wherein compound of formula (III) has a melting point below 50° C.

23. A method for synthesising a compound having the general formula 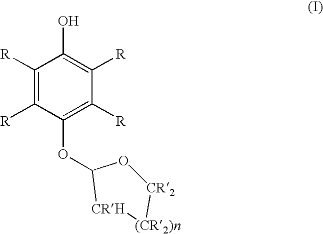
wherein
R is selected independently from the group of H, F, Cl, Br, NO₂, alkyl, aryl, aralkyl, alkoxy, aryloxy, aralkoxy and disubstituted amine;
R′ is selected independently from the group of H, F, Cl, Br, OH, NO₂, alkyl, aryl, aralkyl, alkoxy, aryloxy, aralkoxy and disubstituted amine; and
n is 1, 2, or 3;
comprising
a) reacting a compound of formula (II) in the presence of an acid 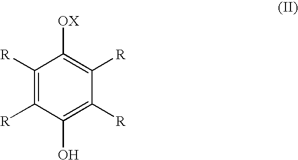
wherein X is a hydroxyl protecting group and R is as defined above
with a compound of formula (III) 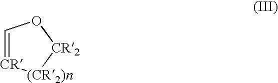
wherein R′ and n are as defined above; and
b) removing the hydroxyl protecting group with hydrogen and a catalyst in the presence of a solvent and a base, said base having a solubility at 20° C. of less than 5% by weight in the solvent.

24. The method of claim 23, wherein said base is selected from the group consisting of alkali and earth alkali carbonates.

25. The method of claim 23, wherein said base is selected from the group consisting of CaCO₃, NaHCO₃, KHCO₃, Na₂CO₃, and K₂CO₃.

26. The method of claim 23, wherein said solvent is selected from the group consisting of alcohols, ether, and esters.

27. The method of claim 26, wherein said solvent is methanol, ethanol, isopropanol, tetrahydrofuran, diethyl ether, methyl tetrabutyl ether, ethyl acetate, butyl acetate, or mixtures thereof.

28. The method of claim 23, wherein said catalyst is selected from the group consisting of precious metals.

29. The method of claim 23, wherein said catalyst is Pd/C.

30. A method for synthesising a compound having the general formula (I) 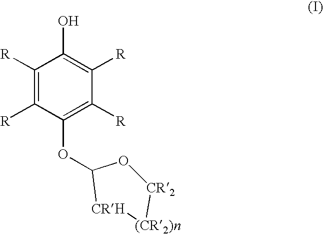
wherein
R is selected independently from the group of H, F, Cl, Br, NO₂, alkyl, aryl, aralkyl, alkoxy, aryloxy, aralkoxy, and disubstituted amine;
R′ is selected independently from the group of H, F, Cl, Br, OH, NO₂, alkyl, aryl, aralkyl, alkoxy, aryloxy, aralkoxy, and disubstituted amine; and
n is 1,2,or3;
comprising
a) reacting a compound of formula (II) in the presence of an acid 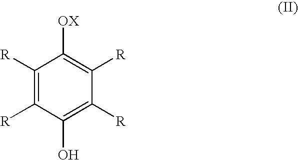
wherein X is a hydroxyl protecting group and R is as defined above with a compound of formula (III) 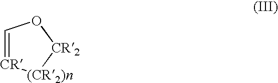
wherein R′ and n are as defined above together with less than 50% by weight of a solvent; and
b) removing the hydroxyl protecting group with hydrogen and a catalyst in the presence of a solvent and a base, said base having a solubility at 20° C. of less than 5% by weight in the solvent.
